(19) United States
(12) Patent Application Publication
(10) Pub. No.: US 2005/0178381 A1
(43) Pub. Date: Aug. 18, 2005

(54) Apparatus and method for humidification of inspired gases

(76) Inventor: Roger Daugherty, Decatur, GA (US)

Correspondence Address: MYERS & KAPLAN, INTELLECTUAL PROPERTY LAW, L.L.C., 1899 POWERS FERRY ROAD, SUITE 310, ATLANTA, GA, 30339, US

(21) Appl. No.: 11/040,179

(22) Filed: Jan. 21, 2005

Related U.S. Application Data
(63) Continuation-in-part of application No. 10/669,108, filed on Sep. 23, 2003.
Application No. 10/669,108 is a continuation-in-part of application No. 10/436,535, filed on May 13, 2003.

(51) Int. Cl. A61M 15/00; A61M 16/10

(52) U.S. Cl. 128/203.12

(57) ABSTRACT

An apparatus and method for humidification of inspired gases, wherein the present invention utilizes moisture from condensed expiratory gases deposited within the outer expiratory tube of a conventional unilimb breathing circuit to humidify oxygen gas (or any other inspiratory gas) for subsequent patient inhalation, and wherein the oxygen gas may be directed through the outer expiratory tube via a flow distributor coupled to an oxygen gas source. The present invention preferably functions to effectively eliminate prior art methods of oxygen gas humidification that depend upon the wasteful utilization of bottles of sterile water, corrugated tubing, nebulizer adapters and excess consumption of oxygen gas; thus, effectuating a cost savings for the patient and contributing to overall environmental conservation efforts.

### CROSS-REFERENCE AND PRIORITY CLAIM TO RELATED APPLICATIONS

To the fullest extent permitted by law, the present continuation-in-part patent application claims priority to and the full benefit of continuation-in-part patent application entitled “Apparatus and Method For Humidification of Inspired Gases”, filed on Sep. 23, 2003, having assigned Ser. No. 10/669,108, which is a continuation-in-part patent application of nonprovisional patent application entitled “Apparatus and Method For Humidification of Inspired Gases”, filed on May 13, 2003, having assigned Ser. No. 10/436,535.

### TECHNICAL FIELD

The present invention relates generally to artificial ventilation systems, and more specifically to an apparatus and method for humidification of inspired gases, wherein the present invention is particularly suitable for, although not strictly limited to, the administration of humidified oxygen gas to patients recovering in a post-anesthesia care unit of a medical facility.

### BACKGROUND OF THE INVENTION

Breathing circuits are commonly utilized in the operating room of a medical facility to convey anesthesia or inspiratory gases from an anesthesia machine to a patient, and to route expiratory gases from the patient to the anesthesia machine for subsequent cleansing and processing of same.

At present, several varieties of breathing circuits are available. One type of breathing circuit of substantial prevalence, and of particular relevance to the present invention as described herein, is a unilimb breathing circuit, wherein examples of such unilimb breathing circuits may be seen with reference to U.S. Pat. No. 4,265,235 to Fukunaga, U.S. Pat. No. 5,404,873 to Leagre et al., and U.S. Pat. No. 6,439,231 to Fukunaga et al. Generally, and as disclosed in the aforementioned patents, unilimb breathing circuits typically comprise a corrugated outer expiratory tube coaxially arranged about a corrugated inner inspiratory tube; that is, a tube-within-a-tube configuration. As such, one end of the unilimb breathing circuit, commonly referred to as the patient end, receives a connector for adapting the unilimb breathing circuit to a face mask, endotracheal tube, or laryngeal tube connected to the patient. The opposing end of the unilimb breathing circuit, commonly referred to as the machine end, typically receives a manifold for adapting the unilimb breathing circuit to an anesthesia machine for requisite inspiratory and expiratory gas manipulation.

Specifically, the manifold functions to direct anesthetic inspiratory gases from the anesthesia machine through the inner inspiratory tube for subsequent patient inhalation. During patient exhalation, expiratory gases flow through the outer expiratory tube and are redirected by the manifold to a carbon dioxide absorber of the anesthesia machine for subsequent removal of carbon dioxide gases therefrom. The cleansed exhaled gases may then be routed back through the inspiratory tube for rebreathing by the patient in conjunction with freshly administered anesthetic inspiratory gases.

In addition to the ability of unilimb breathing circuits to effectively bi-directionally conduct inspiratory and expiratory gases, unilimb breathing circuits are further capable of warming inherently lower temperature anesthesia gases. Essentially, patient expired gases flowing through the outer expiratory tube warm the inherently cooler anesthesia gases flowing through the inner inspiratory tube.

However, as a result of the temperature differential between the inspiratory and expiratory gases, moisture carried within the expiratory gases begins to condense within the corrugations of the expiratory tube, resulting in significant accumulation of moisture therewithin. Although such moisture may provide the ancillary benefit of humidifying the upper respiratory track of the patient during inspiration of dry anesthetic inspiratory gases, the moisture-laden unilimb breathing circuit is typically discarded after its first use, as medical practitioners have been unable to devise a secondary application for the moisture accumulated therewithin.

Discarding the breathing circuit presents the obvious ramification of excess waste of medical supplies, especially in view of the number of medical procedures requiring administration of anesthesia gases, and thus, the use of breathing circuits. Unfortunately, the cost of such expensive medical supplies is often imparted to the patient, adding to an often already overwhelming medical bill.

However, excess use and waste of medical supplies is not limited to disposal of the breathing circuits alone. Following completion of an operation or similar procedure requiring the administration of anesthesia gases via the breathing circuit, the patient is then typically transported from the operating room to the post-anesthesia care unit (i.e., PACU), where the patient is administered fresh oxygen gas to counteract the sedative effects of the anesthesia gases. Prior to patient inhalation of the oxygen gas, however, the inherently dry oxygen gas, delivered via a central oxygen source, must first pass through a bottle of sterile water for purposes of humidifying same, wherein the oxygen gas flow rate is regulated via a conventional flow meter. The humidified oxygen gas is then conveyed to the patient via a second, new length of tubing (i.e., corrugated tubing) connected to a conventional face tent worn by the patient.

Although the above-referenced method provides for the requisite humidification of oxygen gas, it possesses inherent disadvantages that make its implementation highly inefficient and uneconomical. More specifically, the patient is now further responsible for payment of the additional corrugated tubing, the bottle of sterile water, and the associated nebulizer adapter, typically utilized to atomize inspiratory gases passing therethrough. Furthermore, because the oxygen gas must first be passed through the gas-permeable “barrier” of sterile water for humidification purposes (i.e., bottle of sterile water), a higher quantity or percentage of oxygen gas must be passed into the bottle of sterile water to yield an overall effective percentage of humidified oxygen gas suitable for patient inhalation. As such, the patient is also responsible for payment of seemingly unavoidable excess quantities of oxygen gas.

Additionally, in view of efforts to develop products and/or processes that materially contribute to the environmental restoration and/or maintenance of basic life-sustaining natural elements, and the more efficient utilization and conservation of energy resources, the above-discussed method of oxygen gas humidification significantly hinders such present environmental conservation efforts. Specifically, because the bottle of sterile water, corrugated tubing, nebulizer, and associated adaptors and/or accessories, are discarded after first use, millions of gallons of precious water, and valuable petroleum resources utilized to manufacture the plastic tubing, bottle, nebulizer, and the like, are consumed to ensure the sustained provision of such medical supplies.

Therefore, it is readily apparent that there is a need for an apparatus and method for humidification of inspired gases, wherein said apparatus and method utilizes condensed expiratory gases deposited within a breathing circuit to humidify oxygen gas for subsequent patient inhalation, and wherein said apparatus and method functions to effectively eliminate dependency upon prior art methods of humidification, wasteful utilization of bottles of sterile water, corrugated tubing, nebulizer adapters and excess consumption of oxygen gas; thus, effectuating a cost savings for the patient and contributing to overall environmental conservation efforts.

### BRIEF SUMMARY OF THE INVENTION

Briefly described, in a preferred embodiment, the present invention overcomes the above-mentioned disadvantages and meets the recognized need for such a device by providing an apparatus and method for humidification of inspired gases, wherein the present invention utilizes condensed expiratory gases deposited within the outer expiratory tube of a conventional unilimb breathing circuit to humidify oxygen gas (i.e., or any other inspiratory gas) for subsequent patient inhalation, and wherein the oxygen gas is now directed through the outer expiratory tube via a flow distributor coupled to an oxygen gas source. The present invention preferably functions to effectively eliminate prior art methods of oxygen gas humidification that depend upon the wasteful utilization of bottles of sterile water, corrugated tubing, nebulizer adapters and excess consumption of oxygen gas; thus, effectuating a cost savings for the patient and contributing to overall environmental conservation efforts.

According to its major aspects and broadly stated, the present invention in its preferred form is an apparatus and method for humidification of inspired gases comprising a flow distributor, breathing circuit adapter, and cannula tubing adapter, wherein the present inventive apparatus is preferably utilized in conjunction with a unilimb breathing circuit, face tent, and appropriate gas source to deliver humidified inspiratory gases to a patient. Preferably, the flow distributor may be utilized to selectively divert gas flow through the breathing circuit adapter or, alternatively, the cannula tubing adapter.

More specifically, the present invention is an apparatus and method for humidification of inspired gases, wherein the same unilimb breathing circuit utilized to deliver anesthesia gases to a patient in the operating room, is now also utilized to administer humidified oxygen to the same patient transported to and recovering in the PACU, thereby eliminating conventional use of a separate corrugated tubing, bottle of sterile water and nebulizer adapter.

As addressed earlier, during administration of anesthesia gases to the patient in the operating room or the like, patient expired gases flowing through the outer expiratory tube warm the inherently cooler anesthesia gases flowing through the inner inspiratory tube of the unilimb breathing circuit. As a result of the temperature differential between the inspiratory and expiratory gases, moisture carried within the expiratory gases begins to condense within the corrugations of the expiratory tube, resulting in accumulation of moisture therewithin.

Preferably, the accumulated moisture within the expiratory tube is now utilized to humidify the oxygen gas administered to the patient in the PACU; thus, eliminating conventional use of a bottle of sterile water and related accessories.

Preferably, the unilimb breathing circuit utilized for the patient within the operating room now travels with the patient to the PACU, where it is coupled to a central oxygen source via the present flow distributor. That is, the flow distributor is preferably coupled to an oxygen gas source, wherein the breathing circuit adapter of the flow distributor is preferably engaged to the outer expiratory tube of the unilimb breathing circuit. As such, the breathing circuit adapter permits fresh oxygen gas to now travel through the outer expiratory tube of the unilimb breathing circuit (i.e., concentrically about the outside of the inner inspiratory tube), wherein the oxygen gas interacts with the condensed expiratory gases therewithin, picking up moisture therefrom and becoming humidified.

Alternatively, the cannula tubing adapter of the flow distributor may be utilized to assist in the delivery of oxygen gas to a patient when a breathing circuit is unavailable and/or was not utilized in the operating room, and therefore did not accompany the patient to the PACU. Such scenarios may arise when the patient is subjected to modified anesthesia control, wherein anesthesia is delivered intravenously, instead of through a facemask for subsequent inhalation (as a gas). Accordingly, the cannula tubing adapter preferably engages the connector and communicating tube of a standard nasal cannula assembly or simple facemask assembly; thus, enabling the flow of oxygen gas therethrough for subsequent patient inhalation.

Preferably, by eliminating conventional use of a bottle of sterile water and associated accessories for oxygen gas humidification, and by strategically directing oxygen gas flow through the expiratory tube for maximum interaction with condensed expiratory gases therewithin, a lower quantity of oxygen gas (i.e., as drawn from a central oxygen source) can be utilized to deliver an effective percentage of humidified oxygen gas suitable for patient inhalation.

The present invention further contemplates eliminating use of conventional face tents utilized on patients for humidified oxygen gas inhalation. Currently, face tents possessing a standard 22 mm diametered male adapter are commonly utilized, wherein the 22 mm diametered male adapter is coupled to a piece of corrugated tubing having a slightly larger diametered opening to facilitate frictional engagement therewith. Accordingly, the present invention contemplates the manufacture and use of a face tent having a 15 mm diametered male adapter for direct coupling of conventional unilimb breathing circuits thereto. Because most unilimb breathing circuits are commonly manufactured such that the outer expiratory tube possesses a connector or adapter having a diameter sufficient to frictionally engage a 15 mm male connector or adapter of a selected item, conventional corrugated tubing, and associated 22 mm male adapter face tents, utilized for humidified oxygen gas delivery can now be rendered largely extraneous in view of the present invention. However, it is recognized that any suitable face tent having any diametered male or female adapter could be cooperatively engaged to any unilimb breathing circuit having an opening or adapter with an accommodating diameter.

Accordingly, a feature and advantage of the present invention is its ability to humidify oxygen gas via use of condensed water from expiratory gases that accumulate within the expiratory tube of a unilimb breathing circuit.

Another feature and advantage of the present invention is its ability to effectively eliminate prior art methods of oxygen gas humidification that depend upon the wasteful utilization of bottles of sterile water, other corrugated tubing, nebulizer adapters and excess consumption of oxygen gas.

Still another feature and advantage of the present invention is its ability to materially contribute to the environmental restoration and/or maintenance of basic life-sustaining natural elements by eliminating the use of bottles of sterile water for oxygen gas humidification, thus effectively saving millions of gallons of water per year.

Yet another feature and advantage of the present invention is its ability to materially contribute to the more efficient utilization and conservation of energy resources by conserving valuable petroleum resources that would otherwise be utilized to manufacture plastic corrugated tubing, plastic bottles for containing sterile water, and plastic nebulizer adapters, elements crucial to implementation of prior art methods of inspired gas humidification.

Still yet another feature and advantage of the present invention is its ability to effectuate a cost savings for the patient by reducing overuse of medical supplies.

A further feature and advantage of the present invention is that the same unilimb breathing circuit utilized to deliver anesthesia gases to a patient in the operating room is now also utilized to administer humidified oxygen to the same patient transported to and recovering in the PACU.

Still a further feature and advantage of the present invention is that, in comparison to prior art methods of oxygen gas humidification, a lower quantity of oxygen gas (i.e., as drawn from a central oxygen source) can now be utilized to deliver an effective percentage of humidified oxygen gas suitable for patient inhalation.

Yet a further feature and advantage of the present invention is its ability to be implemented with fewer connections and to reduce the likelihood of gas or liquid leaks as compared to prior art methods and devices.

Still yet a further feature and advantage of the present invention is its ability to eliminate operational noises typically associated with conventional apparatuses and methods of gas humidification, wherein the whisper quite operation of the present invention assists clinicians in the accurate and noise-free assessment of breathing sounds or patterns relevant to a patient's clinical state of recovery.

Another and further feature and advantage of the present invention is its ability to filter inspiratory oxygen gas and patient expiratory gases during delivery of humidified oxygen gas to a recovering patient, as opposed to filterless prior art apparatuses and methods of oxygen gas humidification and delivery.

These and other features and advantages of the present invention will become more apparent to one skilled in the art from the following description and claims when read in light of the accompanying drawings.

### BRIEF DESCRIPTION OF THE DRAWINGS

The present invention will be better understood by reading the Detailed Description of the Preferred and Alternate Embodiments with reference to the accompanying drawing figures, in which like reference numerals denote similar structure and refer to like elements throughout, and in which:

FIG. 1 is a front view of an apparatus for humidification of inspired gases according to a preferred embodiment of the present invention;

FIG. 2 is a perspective view of an apparatus for humidification of inspired gases according to a preferred embodiment of the present invention;

FIG. 3 is a side view of an apparatus for humidification of inspired gases according to a preferred embodiment of the present invention FIG. 4 is a partial cross-sectional view of FIG. 3 along section line A-A;

FIG. 5 is front view of an apparatus for humidification of inspired gases according to a preferred embodiment of the present invention, shown in combined use with a unilimb breathing circuit and a face mask;

FIG. 6 is a cross-sectional view of FIG. 5 along section line 1-1;

FIG. 7 is a partial cross-sectional view of FIG. 5 along section line 2-2;

FIG. 8 is a front view of an apparatus for humidification of inspired gases according to an alternate embodiment of the present invention; and, FIG. 9 is a front view of an apparatus for humidification of inspired gases according to an alternate embodiment of the present invention.

### DETAILED DESCRIPTION OF THE PREFERRED AND SELECTED ALTERNATE EMBODIMENTS

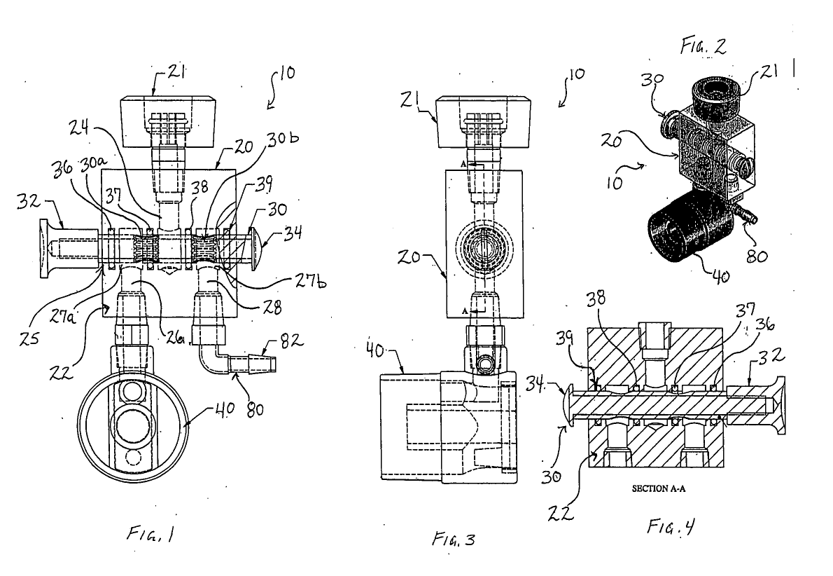

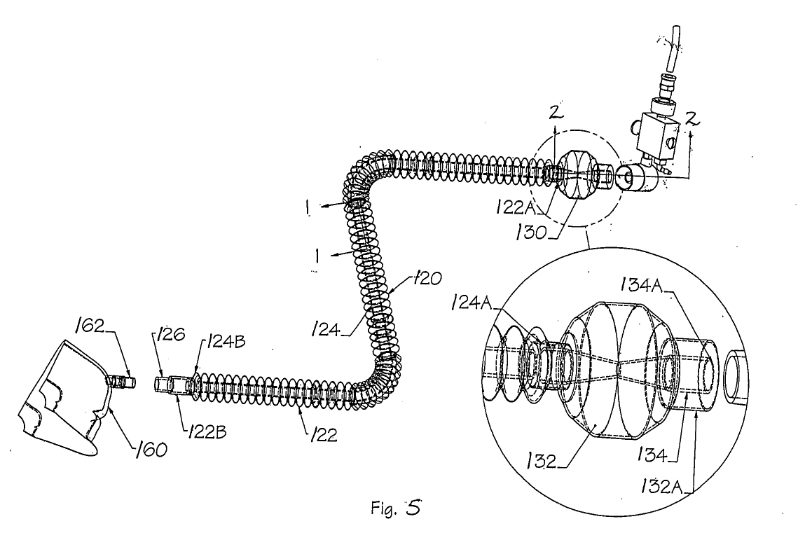

In describing the preferred and selected alternate embodiments of the present invention, as illustrated in FIGS. 1-9, specific terminology is employed for the sake of clarity. The invention, however, is not intended to be limited to the specific terminology so selected, and it is to be understood that each specific element includes all technical equivalents that operate in a similar manner to accomplish similar functions.

Referring now to FIGS. 1-7, the present invention in a preferred embodiment is an apparatus 10, and associated method, for humidification of inspired gases, wherein apparatus 10 preferably generally comprises flow distributor 20, breathing circuit adapter 40, and cannula tubing adapter 80, wherein apparatus 10 is preferably utilized in conjunction with unilimb breathing circuit 120, face tent 160, and central oxygen gas source OGS to deliver humidified oxygen gas to a patient

Referring now more specifically to FIGS. 1-4, flow distributor 20 comprises manifold 22, wherein manifold 22 preferably comprises main flow channel 24, and wherein main flow channel 24 preferably T-intersects, and resides in fluid communication with, central flow channel 25. Central channel 25 preferably branches into, and resides in fluid communication with, primary channel 26 and secondary channel 28. Preferably, breathing circuit adapter 40, the structure of which is more fully described below, resides in fluid communication with primary channel 26. Similarly, cannula tubing adapter 80, the structure of which is also more fully described below, preferably resides in fluid communication with secondary channel 28.

Manifold 22 preferably further comprises flow meter coupler 21 residing in fluid communication with main flow channel 24, wherein threaded engagement of conventional flow meter FM thereto, and subsequent engagement of flow meter FM to central oxygen gas source OGS preferably permits oxygen gas to flow therefrom, through flow meter FM, through main flow channel 24, through central channel 25 and, thereafter, through either primary channel 26 or secondary channel 28, depending upon manual selection of same.

That is, preferably extending through, and slidingly engaged with, central channel 25 is rod-shaped shunting or diverter valve 30, wherein diverter valve 30 is preferably utilized to divert inspiratory gas, oxygen or otherwise, through either primary channel 26 or secondary channel 28, wherein diversion of such gas through primary channel 26 via manual positional manipulation of diverter valve 30 necessarily results in the shunting of secondary channel 28, and vice versa. Preferably, diverter valve comprises grooved regions 30a, 30b, each having a cross-sectional diameter less than the general diameter of diverter valve 30. Accordingly, diverter valve 30 may be slidably manipulated through central channel 25 such that grooved region 30a is positioned to reside substantially over intersecting region 27a of central channel 25 and primary channel 26, wherein such positioning will naturally result in grooved region 30b residing askew relative to intersecting region 27b of central channel 25 and secondary channel 28; thereby, shunting secondary channel 28. In such a configuration, inspiratory gas, oxygen or otherwise, delivered from central oxygen gas source OGS is permitted to ultimately flow through main flow channel 24, through central channel 25, past and over grooved region 30a of diverter valve 30, through primary channel 26 and, thereafter, through breathing circuit adapter 40 for subsequent humidification via the outer expiratory tube of unilimb breathing circuit 120, as more fully described below.

Alternatively, diverter valve 30 may be slidably manipulated through central channel 25 such that grooved region 30b is positioned to reside substantially over intersecting region 27b of central channel 25 and secondary channel 28, wherein such positioning will similarly naturally result in grooved region 30a residing askew relative to intersecting region 27a of central channel 25 and primary channel 26; thereby, shunting primary channel 26. In such a configuration, inspiratory gas, oxygen or otherwise, delivered from central oxygen gas source OGS is permitted to ultimately flow through main flow channel 24, through central channel 25, past and over grooved region 30b of diverter valve 30, through secondary channel 28 and, thereafter, through cannula tubing adapter 80 for subsequent patient inhalation through a standard nasal cannula assembly or simple facemask assembly removably secured thereto.

As is best illustrated in FIG. 1, to assist in slidable movement of diverter valve 30 within central channel 25, diverter valve 30 preferably comprises knob 32. Moreover, diverter valve 30 further comprises stopper or retaining end 34, which, in conjunction with knob 32, functions to prevent over-extension or over-retraction (i.e., complete withdrawal) of diverter valve 30 through or from central channel 25. Additionally, to prevent escape of inspiratory gas past intersecting region 27a of central channel 25 and primary channel 26, and intersecting region 27b of central channel 25 and secondary channel 28, sealant rings or O-rings 36, 37 and 38, 39 are preferably disposed within central channel 25, and flank respective intersecting regions 27a, 27b. Although O-rings 36, 37, 38, 39 are the preferred form of sealant, it should be recognized that any suitable sealant may be utilized, such as, for exemplary purposes only, gaskets, washers, flat rings, silicone, rubber sealants, and the like.

Referring now more specifically to FIGS. 5-7, with continued reference to FIGS. 1-4, unilimb breathing circuit 120 is preferably comparable to those disclosed in U.S. Pat. No. 4,265,235 to Fukunaga, U.S. Pat. No. 5,404,873 to Leagre et al., and U.S. Pat. No. 6,439,231 to Fukunaga et al. and, as such, is preferably utilized to administer anesthesia gases to a patient undergoing a surgical operation, or other medical procedure requiring patient sedation, and is further preferably utilized to convey expiratory gases away from the patient. Preferably, unilimb breathing circuit 120 possesses corrugated outer expiratory tube 122 coaxially arranged about corrugated inner inspiratory tube 124. Expiratory tube 122 preferably includes machine end 122a and patient end 122b, wherein inspiratory tube 124 also preferably includes machine end 124a and patient end 124b, respectively positioned proximal to ends 122a, 122b of expiratory tube 122.

Preferably, coaxial filter 130 is in fluid communication with machine ends 122a, 124a of expiratory tube 122 and inspiratory tube 124, respectively. More specifically, outer port 132 of coaxial filter 130 is in fluid communication with machine end 122a of expiratory tube 122, wherein inner port 134 is preferably in fluid communication with machine end 124a of inspiratory tube 122. As more fully described below, ends 132a, 134a of outer port 132 and inner port 134, respectively, preferably cooperatively engage breathing circuit adapter 40 of flow distributor 20 for implementation of the present method of humidification of inspired gas. As is known within the art, coaxial filter 130 is preferably any suitable coaxial filter capable of being adapted to any conventional unilimb breathing circuit, and is preferably utilized to reduce and/or prevent bacterial transmission via suitable filter mediums such as, for exemplary purposes only, high efficiency particulate assembly (H.E.P.A.) filters.

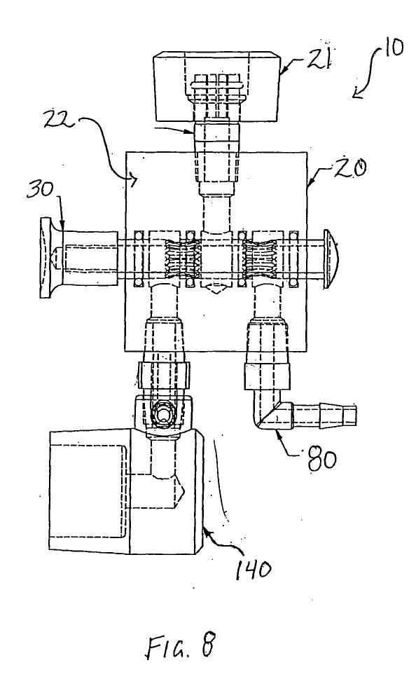

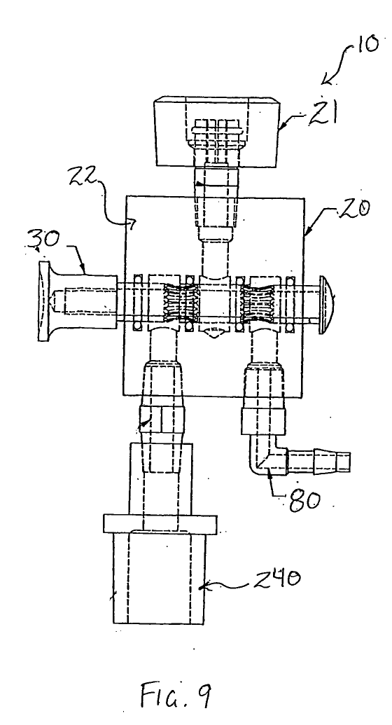

Although unilimb breathing circuit 120, comparable to those disclosed in U.S. Pat. No. 4,265,235 to Fukunaga, U.S. Pat. No. 5,404,873 to Leagre et al., and U.S. Pat. No. 6,439,231 to Fukunaga et al., is preferably utilized to implement the present method of inspired gas humidification, it is contemplated in an alternate embodiment that other suitable breathing circuits could be utilized without departing from the appreciative scope of the present invention, so long as the selected breathing circuit contributes to the accumulation of condensed expiratory gases therewithin; such as, for exemplary purposes only, other types of unilimb breathing circuits, suitable dual-limb breathing circuits, filtered breathing circuits, unfiltered breathing circuits, corrugated breathing circuits and/or non-corrugated breathing circuits, wherein such alternate forms of breathing circuits are in full contemplation of the inventor in describing the present invention herein. Indeed, such alternate breathing circuits, in combined use with flow distributor 20, for implementation of the present method of humidification of inspired gas, are more fully described with reference to FIGS. 8-9 hereinbelow.

Preferably patient end 122b of expiratory tube 122 comprises connector 126 in communication therewith, wherein connector 126 is preferably appropriately dimensioned to facilitate frictional engagement of male adapter 162 of face tent 160 therewith, as more fully developed below.

Procedurally, and as known within the art, a patient undergoing a medical procedure requiring patient sedation is typically administered sedative or anesthetic gases via coupling of breathing circuit 120 to an anesthesia machine. Specifically, machine ends 122a, 122b of breathing circuit 120 receive a manifold (not shown) for adapting breathing circuit 120 to an anesthesia machine (not shown) for requisite inspiratory and expiratory gas manipulation. Connector 126 of patient end 122b of expiratory tube 122 of breathing circuit 120 is coupled to an adapter (not shown) to facilitate engagement of a face mask, endotracheal tube, or laryngeal tube (not shown) thereto, wherein the face mask, or the like, is worn by the patient to facilitate inhalation of the anesthetic gases.

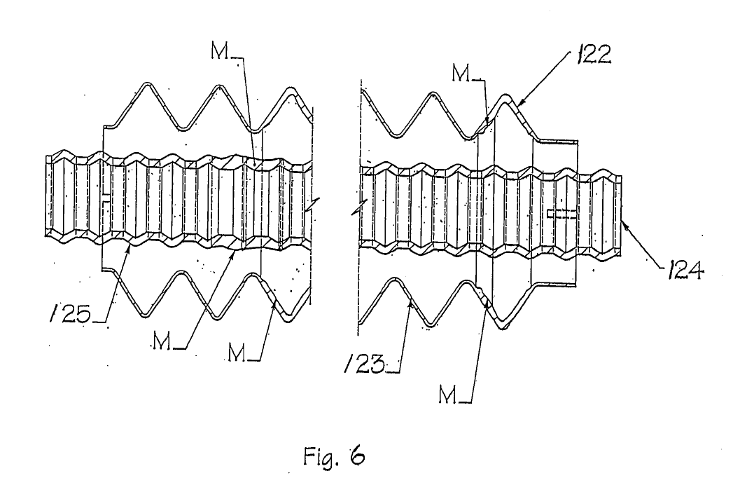

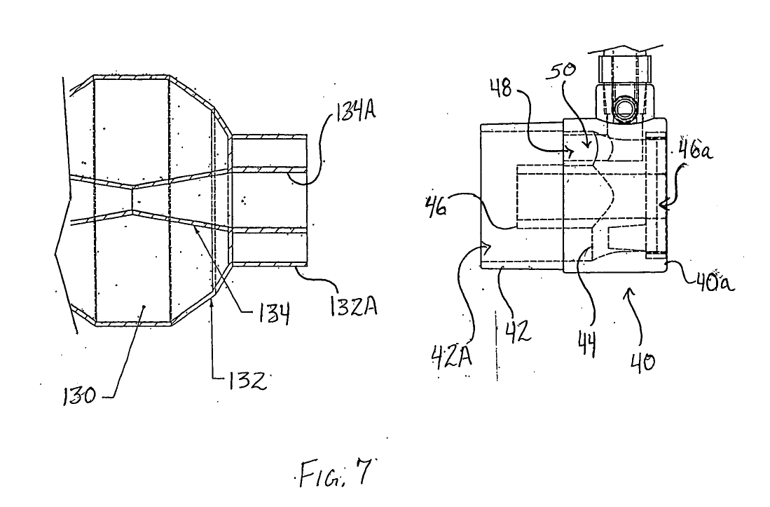

During such anesthetic gas administration to the patient, the patient's expired gases flow through expiratory tube 122 and warm the inherently cooler anesthesia gases flowing through inspiratory tube 124 of unilimb breathing circuit 120. As a result of the temperature differential between the inspiratory and expiratory gases, moisture carried within the patient's expiratory gases begins to condense within corrugations 123 of expiratory tube 122 and on the outer surface of corrugations 125 of inspiratory tube 124; thus, resulting in accumulation of moisture M therewithin, as best illustrated in FIG. 6. As more fully described below, the present method preferably utilizes moisture M and breathing circuit 120 to humidify the inherently dry oxygen gas (or other inspired gases) administered to the patient in the PACU; thereby, eliminating conventional use of a bottle of sterile water, corrugated tubing and nebulizer adapter.

Following completion of the medical procedure, and cessation of anesthesia gas administration, the patient is then typically (procedurally) transported from the operating room to the PACU, where the patient is administered fresh oxygen gas to counteract the sedative effects of the anesthesia gases. Generally, conventional methods of oxygen gas administration require the use of a new length or piece of corrugated tubing and a face tent (or endotracheal tube or laryngeal tube), because the moisture saturated (i.e., condensed expiratory gases) breathing circuit previously utilized for anesthesia gas administration has been discarded.

However, the present apparatus and method preferably seeks to utilize the moisture M saturated breathing circuit 120 to humidify the inherently dry oxygen gas (or other inspired gases) administered to the patient in the PACU; thereby, eliminating the conventional and uneconomical use of a bottle of sterile water, new corrugated tubing, and nebulizer adapter, for oxygen gas humidification.

Preferably, unilimb breathing circuit 120 and coaxial filter 130, along with accumulated moisture M still retained within expiratory tube 122, are transported with the patient to the PACU, wherein breathing circuit 120 is subsequently preferably coupled to central oxygen gas source OGS via flow distributor 10 as described hereinabove, and as further described hereinbelow.

Referring now more specifically to FIG. 7, illustrated therein is a cross-sectional view of breathing circuit adapter 40 and coaxial filter 130, wherein breathing circuit adapter 40 is preferably in fluid communication with primary channel 26 of flow distributor 20. Preferably breathing circuit adapter 40 comprises inlet 42 defining recessed area 42A, wherein inlet 42 comprises a circumference sufficient to be frictionally received and engaged within end 132a of outer port 132 of coaxial filter 130. Centrally formed and extending from rear wall 44 of recessed area 42a is hollowed protuberance 46, defining passageway 46a extending therethrough, and exiting out from anterior side 40a of adapter 40. Protuberance 46 is dimensioned to be frictionally received and engaged within end 134a of inner port 134 of coaxial filter 130, for purposes more fully described below.

Formed through rear wall 44 of recessed area 42a, and positioned above hollowed protuberance 46, is aperture 48, wherein aperture 48 is in fluid communication with substantially L-shaped passageway or channel 50. Aperture 48 and channel 50 are positioned above hollowed protuberance 46 so as to not cross-sect and interrupt passageway 46a, for purposes more fully described below. Channel 50 and aperture 48 function to permit flow of oxygen gas therethrough, as delivered via conventional flow meter FM and central oxygen gas source OGS through flow distributor 20, for subsequent channeling of same through expiratory tube 122.

More specifically, upon slidably engaging end 132a of outer port 132 of coaxial filter 130 over inlet 42 of breathing circuit adapter 40, end 134a of inner port 134 of coaxial filter 130 frictional receives and engages protuberance 46 of breathing circuit adapter 40, thus bringing passageway 46a thereof in fluid communication with inner port 134 and communicating inspiratory tube 124. As described above, oxygen gas delivered via central oxygen gas source OGS, flows through conventional flow meter FM, through main flow channel 24 of flow distributor 10, through central channel 25, past and over grooved region 30a of diverter valve 30, through primary channel 26, through aperture 48 of breathing circuit adapter 40, through outer port 132 of coaxial filter 130, and through expiratory tube 122, for subsequent interaction with, and humidification by, moisture M accumulated therewithin.

That is, as oxygen gas travels through expiratory tube 122 (i.e., on the outside of inspiratory tube 124) in a natural toroidal and/or helical manner toward patient end 122b thereof, the oxygen gas preferably interacts with accumulated moisture M (i.e., condensed expiratory gases) deposited within corrugations 123 of expiratory tube 122 and on the outer surface of corrugations 125 of inspiratory tube 124, picking up moisture therefrom and thus, becoming humidified.

Preferably, when the oxygen gas traveling through expiratory tube 122 reaches patient end 122b thereof, the oxygen gas is preferably sufficiently humidified for patient inhalation, wherein the humidified oxygen gas preferably exits patient end 122b through a conventional connector 126.

Connector 126 preferably possesses an appropriately dimensioned diameter to facilitate frictional engagement of male adapter 162 of face tent 160 therewith. Preferably, male adapter 162 possesses a diameter of approximately 15 mm for the direct frictional coupling of connector 126 of unilimb breathing circuit 120 thereto, as connector 126 is typically (conventionally) manufactured to possess an inner diameter of approximately 15 mm. As stated earlier, because most unilimb breathing circuits are commonly manufactured such that the outer expiratory tube possesses a connector or adapter having a diameter sufficient to frictionally engage a 15 mm male connector or adapter of a selected item, conventional corrugated tubing, and associated 22 mm male adapter face tents, utilized for humidified oxygen gas delivery, can now be rendered largely extraneous in view of the present invention. However, it is recognized that any suitable face tent having any diametered male or female adapter could be cooperatively engaged to any unilimb breathing circuit having an opening or connector with an accommodating diameter, wherein such dimensions and/or configurations could be utilized without departing from the appreciate scope of the present invention, and are in full contemplation of the inventor in describing the present invention herein.

To facilitate delivery of such humidified oxygen gas to intubated patients (i.e., patients fitted with an endotracheal tube or laryngeal tube), connector 126 engaged with patient end 122b of expiratory tube 122 is connected to a conventional adapter formed at the end of the endotracheal tube or laryngeal tube extending out from the intubated patient, thereby permitting the flow of humidified oxygen gas therethrough.

However, as the natural process of inhalation necessitates subsequent exhalation, intubated patients inhaling or receiving oxygen gas must be supplied with a method or avenue to exhale waste gases. Conventional practice requires the attachment of a T-tube or T-piece to the end of the endotracheal tube or laryngeal tube extending out from the patient, wherein a tube carrying oxygen gas may be connected to a first arm thereof. As such, and as known within the art, when an intubated patient exhales, the exhaled gases exit through a second arm of the T-piece, while fresh oxygen gas continues to enter through the first arm thereof.

However, utilization of breathing circuit adapter 40 with unilimb breathing circuit 120 for application to intubated patients advantageously eliminates the need for T-pieces, or the like. Specifically, because passageway 46a of protuberance 46 is in fluid communication with inner port 134 of coaxial filter 130 and communicating inspiratory tube 124, exhaled gases released by an intubated patient travel through inspiratory tube 124, through inner port 134 of coaxial filter 130, through passageway 46a of protuberance 46, and exit through anterior side 40a of breathing circuit adapter 40; while humidified oxygen gas continues to flow through expiratory tube 122 and through a connecting endotracheal or laryngeal tube. It is contemplated in an alternate embodiment that a face tent could be connected to expiratory tube 122 for utilization of breathing circuit adapter 40 with non-intubated patients.

With specific reference now to cannula tubing adapter 80, as described above, diverter valve 30 may be selectively positioned so as to utilize cannula tubing adapter 80 for delivery of oxygen gas to a patient when a breathing circuit is unavailable and/or was not utilized in the operating room, and therefore did not accompany the patient to the PACU. Such scenarios may arise when the patient is subjected to modified anesthesia control, wherein anesthesia is delivered intravenously, instead of through a facemask for subsequent inhalation (as a gas). Accordingly, and as best illustrated in FIG. 1, cannula tubing adapter 80 comprises a tapered connecting end 82 preferably dimensioned to engage the connector and communicating tube of a standard nasal cannula assembly or simple facemask assembly; thus, enabling the flow of oxygen gas therethrough for subsequent patient inhalation.

Referring now more specifically to FIG. 8, illustrated therein is an alternate embodiment of apparatus 10, wherein the alternate embodiment of FIG. 8 is substantially equivalent in form and function to that of the preferred embodiment detailed and illustrated in FIGS. 1-7 except as hereinafter specifically referenced. Specifically, the embodiment of FIG. 8 replaces breathing circuit adapter 40 with breathing circuit adapter 140, wherein adapter 140 is preferably a 22 mm female adapter for cooperative engagement with the terminal end of an expiratory hose of alternate, and commonly available, coaxial breathing circuits.

Referring now more specifically to FIG. 9, illustrated therein is an alternate embodiment of apparatus 10, wherein the alternate embodiment of FIG. 9 is substantially equivalent in form and function to that of the preferred embodiment detailed and illustrated in FIGS. 1-7 except as hereinafter specifically referenced. Specifically, the embodiment of FIG. 9 replaces breathing circuit adapter 40 with breathing circuit adapter 240, wherein adapter 140 is preferably a 22 mm male adapter for cooperative engagement with the expiratory tee of alternate, and commonly available, coaxial breathing circuits.

With general reference now to apparatus 10, and the various preferred and alternate embodiments thereof, including the associated methods of inspiratory gas humidification, by eliminating conventional use of a bottle of sterile water and associated accessories for oxygen gas humidification, and by strategically directing oxygen gas flow through expiratory tube 122 for maximum interaction with accumulated moisture M therewithin, a lower quantity of oxygen gas (i.e., as drawn from central oxygen gas source OGS) can be utilized to deliver an effective percentage of humidified oxygen gas suitable for patient inhalation. Specifically, conventional methods of oxygen gas humidification utilizing a bottle of sterile water for humidification purposes, typically require that the central oxygen gas source OGS maintain an oxygen flow rate of 10 to 12 liters per minute. However, via implementation of the present method of oxygen gas humidification, oxygen flow rates can effectively be reduced to 5 to 6 liters per minute. Additionally, clinical studies and experimental testing conducted by the inventor have established that implementation of the present method of oxygen gas humidification, utilizing an oxygen flow rate of 6 liters per minute, provides the requisite 98% to 100% inspired oxygen level (FiO2) for stabilization of patient blood oxygen saturation.

Although the present apparatus and method is preferably utilized for humidification of oxygen gas, it should be recognized that the present invention could be utilized to humidify any suitable gas and/or combination of gases.

Additionally, although the present method may be implemented with an oxygen flow rate of 6 liters per minute, it is contemplated in an alternate embodiment that either lower or higher oxygen flow rates could be utilized.

It is contemplated in an alternate embodiment that flow distributor 20 could incorporate a flow diluter utilized to ween a patient off oxygen gas as the patient's normal metabolic functions return, and as the sedative effects of anesthesia gases steadily diminish, and wherein such flow diluters are known within the art. Specifically, such a flow diluter could be threadably engaged to coupler 21 of flow distributor 20, wherein flow meter FM could accordingly be coupled thereto. As such, oxygen gas, as delivered via central oxygen gas source OGS, travels through flow meter FM, through the flow diluter, and through flow distributor 20 as described hereinabove. To dilute the percentage of oxygen gas being delivered to a patient (specifically, from 100% to 50%), a rotatable sleeve disposed on the flow diluter could be rotated to expose an aperture formed through the flow diluter, wherein room air would be permitted to enter therethrough, intermix with the metered oxygen gas flowing therethrough (i.e., via a Venturi effect), and dilute the final inhaled and humidified oxygen gas from 100% to 50%. It should be recognized that the flow diluter and associated sleeve and aperture could be modified to permit dilution of oxygen gas to any desired percentage, ranging from 0% to 100%.

It is contemplated in another alternate embodiment that, although breathing circuit adapter 40 comprises aperture 48 alone, breathing circuit adapter 40 could comprise any number of apertures for expelling oxygen gas through expiratory tube 122, wherein the added aperture(s) could be selectively positioned within breathing circuit adapter 40

It is contemplated in still another alternate embodiment that breathing circuit adapter 40 could possess a plurality of apertures concentrically arranged about protuberance 46 for expelling oxygen gas through expiratory tube 122.

It is contemplated in yet another alternate embodiment that flow distributor 20 could be integrally formed with conventional flow meter FM.

It is contemplated in still yet another alternate embodiment that coaxial filter 130 could be entirely eliminated, wherein machine ends 122a, 124a of expiratory tube 122 and inspiratory tube 124, respectively, could be directly coupled to breathing circuit adapter 40.

It is contemplated in a further alternate embodiment that protuberance 46 of breathing circuit adapter 40 could be entirely eliminated; thus, permitting oxygen gas to flow through both expiratory tube 122 and inspiratory tube 124.

It is contemplated in still a further alternate embodiment that other suitable face tents, masks, cannula, tubing, and/or the like could be adapted to the patient for effective inhalation of humidified inspired gases.

It is contemplated in yet a further alternate embodiment that flow distributor 20 and flow meter FM4 could be integrally formed and/or permanently mounted to central oxygen gas source OGS, as each coaxial filter 130 of each unilimb breathing circuit 120 would prevent bacterial or microbial contamination of flow distributor 20 and flow meter FM.

It is contemplated in still yet a further alternate embodiment that ends 132a, 134a of outer port 132 and inner port 134 of coaxial filter 130 could be frictionally received and engaged by breathing circuit adapter 40.

It is contemplated in another and further alternate embodiment that small quantities of sterile water could be introduced into expiratory tube before or during administration of oxygen gas (or other inspired gases) for purposes of maintaining a select quantity of moisture therein.

It is contemplated in still another and further alternate embodiment that primary channel 26 and secondary channel 28 could comprise separate diverter valves to enable the selective or contemporaneous flow of inspiratory gas through either or both primary channel 26 and secondary channel 28, and, thus, respective breathing circuit adapter 40 (or 140, 240) or cannula tubing adapter 80. Such an embodiment would be particularly useful for administering inspiratory gas to two separate patients when only one (oxygen) inspiratory gas source is available. Indeed the present alternate embodiment further contemplates the manufacture of a flow distributor comprising multiple flow channels each in fluid communication with a respective plurality of breathing circuit adapters 40, 140, 240 and/or cannula tubing adapters 80.

It is contemplated in yet another and further alternate embodiment that the present apparatus and method could be implemented during the transport of a patient from one location to another via utilization of a mobile oxygen gas source.

Having thus described exemplary embodiments of the present invention, it should be noted by those skilled in the art that the within disclosures are exemplary only, and that various other alternatives, adaptations, and modifications may be made within the scope of the present invention. Accordingly, the present invention is not limited to the specific embodiments illustrated herein, but is limited only by the following claims.

1. An apparatus for enabling the humidification of inspiratory gas, wherein the inspiratory gas is directed through an expiratory tube of a coaxial breathing circuit, and wherein the inspiratory gas interacts with and becomes humidified by moisture that had previously condensed and accumulated within the expiratory tube during prior delivery of anesthetic gases to a patient through an inspiratory tube of the coaxial breathing circuit, said apparatus comprising:
a flow distributor; and,
a breathing circuit adapter.

2. The apparatus of claim 1, wherein said flow distributor is in fluid communication with said breathing circuit adapter.

3. The apparatus of claim 1, wherein said breathing circuit adapter is adapted to engage the expiratory tube of the coaxial breathing circuit.

4. The apparatus of claim 1, wherein said flow distributor comprises a manifold through which the inspiratory gas may flow.

5. The apparatus of claim 4, wherein said manifold resides in fluid communication with a flow meter coupler, wherein said flow meter coupler is adapted to receive a flow meter for engagement of said flow distributor to an inspiratory gas source.

6. The apparatus of claim 4, wherein said manifold comprises a primary flow distribution channel and a secondary flow distribution channel.

7. The apparatus of claim 6, wherein said primary flow distribution channel is in fluid communication with said breathing circuit adapter.

8. The apparatus of claim 6, wherein said secondary flow distribution channel is in fluid communication with a cannula tubing adapter, said cannula tubing adapter carried by said flow distributor.

9. The apparatus of claim 6, wherein said manifold comprises a diverter valve, and wherein flow of the inspiratory gas through said primary flow distribution channel and said secondary flow distribution channel is controlled by said diverter valve, so as to at least partially shunt flow of the inspiratory gas through either said primary flow distribution channel or said secondary flow distribution channel.

10. The apparatus of claim 9, wherein said diverter valve is slidably engaged through a central flow distribution channel of said manifold, wherein said central flow distribution channel is in fluid communication with said primary flow distribution channel and said secondary flow distribution channel.

11. The apparatus of claim 10, wherein said diverter valve comprises a first and a second grooved region, and wherein lateral positional manipulation of said diverter valve through said central flow distribution channel aligns said first or said second grooved region over respective said primary flow distribution channel or said secondary flow distribution channel to permit the flow of inspiratory gas over either said first or said second grooved region and over respective said primary flow distribution channel or said secondary flow distribution channel.

12. The apparatus of claim 11, wherein said central flow distribution channel comprises sealants to prevent the leakage of inspiratory gas from opposing ends of said central flow distribution channel.

13. The apparatus of claim 12, wherein said central flow distribution channel is in fluid communication with a main flow channel of said manifold, said main flow channel in fluid communication with a flow meter coupler, and wherein said flow meter coupler is adapted to receive a flow meter for engagement of said flow distributor to an inspiratory gas source for delivery of the inspiratory gas through said manifold.

14. An apparatus for enabling the humidification of inspiratory gas, wherein the inspiratory gas is directed through an expiratory tube of a coaxial breathing circuit, and wherein the inspiratory gas interacts with and becomes humidified by moisture that had previously condensed and accumulated within the expiratory tube during prior delivery of anesthetic gases to a patient through an inspiratory tube of the coaxial breathing circuit, said apparatus comprising:
a flow distributor;
a breathing circuit adapter, wherein said breathing circuit adapter resides in fluid communication with said flow distributor, and wherein said breathing circuit adapter is adapted to engage the expiratory tube of the coaxial breathing circuit; and,
a cannula tubing adapter, said cannula tubing adapter residing in fluid communication with said flow distributor.

15. The apparatus of claim 14, wherein said flow distributor comprises a manifold through which the inspiratory gas may flow and exit said breathing circuit adapter and said cannula tubing adapter.

16. The apparatus of claim 15, wherein said manifold comprises a diverter valve, and wherein said diverter valve selectively and at least partially shunts flow of the inspiratory gas through either said breathing circuit adapter or said cannula tubing adapter.

17. The apparatus of claim 14, wherein said cannula tubing adapter comprises a nipple, and wherein said nipple engages a cannula tubing assembly.

18. The apparatus of claim 14, wherein said breathing circuit adapter is adapted to further engage the inspiratory tube of the coaxial breathing circuit and thereby enable patient exhaled gases to travel therethrough and exit through said breathing circuit adapter.

19. A system of humidifying an inspiratory gas delivered from an inspiratory gas source, said system comprising:
a flow distributor, wherein said flow distributor is in fluid communication with the inspiratory gas source, and wherein said flow distributor enables the inspiratory gas to flow therethrough; and,
a breathing circuit adapter in fluid communication with said flow distributor, wherein said breathing circuit adapter is adapted to engage an expiratory tube of a unilimb breathing circuit, wherein the inspiratory gas is directed through said flow distributor and through the expiratory tube, where the inspiratory gas interacts with and becomes humidified by moisture that had previously condensed and accumulated within the expiratory tube during prior delivery of anesthetic gases to a patient through an inspiratory tube of the unilimb breathing circuit.

20. The system of claim 19, wherein said flow distributor further comprises a cannula tubing adapter, and further comprising a diverter valve, and wherein said diverter valve selectively and at least partially shunts flow of the inspiratory gas through either said breathing circuit adapter or said cannula tubing adapter.
